(19) United States
(12) Patent Application Publication
(10) Pub. No.: US 2006/0029551 A1
(43) Pub. Date: Feb. 9, 2006

(54) Stable particle formulations of erythropoietin receptor agonists

(76) Inventors: Kui Liu, Redwood City, CA (US); Michael A. Desjardin, Sunnyvale, CA (US)

Correspondence Address: DEWIPAT INCORPORATED, 4606 FM 1960 WEST, SUITE 400, PMB 166, HOUSTON, TX, 77069, US

(21) Appl. No.: 11/194,889

(22) Filed: Aug. 1, 2005

Related U.S. Application Data
(60) Provisional application No. 60/599,663, filed on Aug. 5, 2004.

(51) Int. Cl. A61K 38/18; A61K 31/7012; A61K 9/14; A61L 9/04

(52) U.S. Cl. 424/46; 530/350; 514/12; 514/53

(57) ABSTRACT

A particle formulation includes an erythropoietin receptor agonist, a buffer, and a sugar, wherein the buffer and sugar stabilize the erythropoietin receptor agonist against aggregation.

### CROSS-REFERENCE TO RELATED APPLICATIONS

This application claims priority from U.S. provisional application No. 60/599,663, filed Aug. 5, 2004, the content of which is incorporated herein by reference.

### BACKGROUND OF THE INVENTION

The invention relates generally to pharmaceutical formulations that are stable at elevated temperature for a long duration.

Erythropoietin (EPO) is a pleiotropic glycoprotein hormone produced primarily by the kidney. EPO stimulates the bone marrow to produce red blood cells and exerts tissue protective effects, e.g., neuroprotection, outside the bone marrow. EPO exerts its biological effect by binding to its cell surface receptor. EPO receptor agonists (ERAs) are a class of recombinant molecules that can activate EPO receptors. The recombinant molecules in the ERA class may or may not contain sequence homology to native human EPO (hEPO). Examples of products in the ERA class containing sequence homology to native hEPO are shown in Table 1 below.

TABLE 1

| | Recombinant | Homology of Amino |
|---|---|---|
| Product Name | Molecule | Acid sequence to hEPO |
| PROCRIT ®/EPOGEN ® | Epoetin alfa | 100% |
| EPREX ®/ERYPO ® | Epoetin alfa | 100% |
| NeoRecormon ® | Epoetin beta | 100% |
| ARANESP ® | Darbepoetin alfa | 97% |

ERA products have been indicated for treatment of anemia due to chronic renal failure, anemia associated with cancer chemotherapy and surgery, and anemia secondary to AZT treatment of AIDS. ERA products currently on the market are administered to patients by subcutaneous or intramuscular injection thrice a week (EPREX®, ERYPO®, and PROCRIT®) or once a week (ARANESP®). The need for these frequent injections could be eliminated if ERAs could be formulated for delivery via sustained release delivery platforms, such as pump implants and depot injections, or non-invasive delivery platforms, such as transdermal patches.

In general, sustained release delivery platforms require formulations that are stable when stored for long durations, e.g., several weeks or months, at elevated temperature, e.g., 37° C. or higher. Several ERA products currently on the market are liquid, are required to be stored at 2 to 8° C., and are unstable at room and elevated temperatures. ERAs are prone to aggregation, which may compromise biological activity and induce unwanted side effects such as immunogenicity.

From the foregoing, there is a desire for ERA formulations that are stable when stored for long durations, e.g., several weeks or months, at elevated temperature, e.g., 37° C. or higher.

### SUMMARY OF THE INVENTION

In one aspect, the invention relates to a particle formulation comprising an erythropoietin receptor agonist, a buffer and a sugar, wherein the buffer and sugar stabilize the erythropoietin receptor agonist against aggregation.

In another aspect, the invention relates to a particle formulation comprising an erythropoietin receptor agonist, a buffer selected from the group consisting of citrate and histidine, and a sugar, wherein the particle formulation has a total soluble aggregate less than 3% over 1 month at 40° C.

Other features and advantages of the invention will be apparent from the following description.

### BRIEF DESCRIPTION OF THE DRAWINGS

FIG. 1A shows effect of pH on stability of EPO in solution.

FIG. 1B shows effect of buffer type on stability of EPO in solution.

FIG. 1C shows effect of NaCl on stability of EPO in solution.

FIG. 1D shows effect of surfactant on stability of EPO in solution.

FIG. 1E shows effect of metal complex on stability of EPO in solution.

FIG. 1F shows effect of arginine on stability of EPO in solution.

FIG. 1G shows effect of sucrose on stability of EPO in solution.

FIG. 2 shows EPO loading at initial, 8 days, and 4 weeks at 40° C., and at 3 months at 37° C. for citrate-buffered lyophilized formulations according to embodiments of the invention.

FIG. 3 shows total soluble aggregate at initial, 8 days, and 4 weeks at 40° C., and at 3 months at 37° C. for citrate-buffered lyophilized formulations according to embodiments of the invention.

FIG. 4 illustrates the effects of sucrose to EPO ratio, surfactant concentration, and buffer concentration on total soluble aggregate for citrate-buffered lyophilized formulations according to embodiment of the invention.

FIG. 5 shows EPO loading at initial, 8 days, and 4 weeks at 40° C., and at 3 months at 37° C. for histidine-buffered lyophilized formulations according to embodiments of the invention.

FIG. 6 shows total soluble aggregates at initial, 8 days, and 4 weeks at 40° C., and at 3 months at 37° C. for histidine-buffered lyophilized formulations according to embodiments of the invention.

### DETAILED DESCRIPTION OF THE INVENTION

The invention will now be described in detail with reference to a few preferred embodiments, as illustrated in accompanying drawings. In the following description, numerous specific details are set forth in order to provide a thorough understanding of the invention. However, it will be apparent to one skilled in the art that the invention may be practiced without some or all of these specific details. In other instances, well-known features and/or process steps have not been described in detail in order to not unnecessarily obscure the invention. The features and advantages of the invention may be better understood with reference to the drawings and discussions that follow.

The invention provides particle formulations of ERA that are stable at elevated temperature for a long duration. For example, particle formulations according to embodiments of the invention are physically and chemically stable at 40° C. for at least 1 month and at 37° C. for at least 3 months (delivery conditions). A particle formulation may be considered to be chemically stable if an acceptable percentage of degradation products produced by chemical pathways such as deamidation (usually by hydrolysis) or oxidation is formed. For example, a formulation may be considered chemically stable if less than 35%, preferably no more than about 20%, breakdown products are formed after 3 months at delivery conditions. A particle formulation may be considered to be physically stable if an acceptable percentage of aggregates (e.g., dimers and other higher molecular weight products) is formed. For example, a formulation may be considered to be physically stable if less than 15%, preferably no more than 10%, more preferably less than 3%, aggregates are formed after 3 months at delivery conditions.

Since stability at an elevated temperature can serve as an accelerated measure of stability at a lower temperature, particle formulations according to embodiments of the invention are also expected to be stable at lower temperatures, such as room temperature and refrigeration temperature. The particle formulations include an ERA stabilized against aggregation with a buffer and a stabilizer including a sugar. The particle formulations may be prepared by lyophilization, spray-drying, or other method available in the art to form particles from a mixture of components. Spray-dried formulations may have an advantage over lyophilized formulations since the process is fast and the particle size is small with narrow distribution so that a further grinding process is not needed. Particle formulations according to embodiments of the invention have a low moisture content, typically less than 5% by weight. Particle formulations according to embodiments of the invention could be suspended in appropriate vehicles for delivery via sustained release or non-invasive delivery platforms.

The term “ERA” or “erythropoietin receptor agonist” refers to a class of recombinant molecules that can activate EPO receptors. These recombinant molecules may or may not contain sequence homology to native hEPO. An ERA according to one embodiment of the invention may be selected from the group consisting of polypeptides and proteins having the biological activity of recombinant hEPO, EPO analogs, EPO isoforms, EPO mimetics, EPO fragments, hybrid EPO proteins, fusion protein oligomers and multimers of the above, homologues of the above, glycosylation pattern variants of the above, muteins of the above, and EPO molecules containing the minor modifications enumerated above. ERAs according to the present invention shall not be limited by method of synthesis or manufacture and shall include those synthesized or manufactured by recombinant (whether produced from cDNA or genomic DNA), synthetic, transgenic, and gene activated methods.

Particularly preferred ERAs are those that are capable of stimulating erythropoiesis in a mammal. Examples of ERAs capable of stimulating erythropoiesis in a mammal include, but are not limited to, epoetin alfa (trade name EPREX®, ERYPO®, PROCRIT®), epoetin beta (trade name NEORECORMON®), and darbepoetin alfa (trade name NESP™, ARANESP®). One form of darbepoetin alfa is described in PCT Publication WO 95/05465 (Amgen, Inc.), the tutorial content of which is incorporated herein by reference. In the WO 95/05465 publication, a darbepoetin alfa includes an analog of hEPO comprising an amino acid sequence which includes at least one additional site or a rearrangement of at least one site for glycosylation. The glycosylation site is for an N-linked or O-linked carbohydrate chain.

Other ERAs indicated as capable of stimulating erythropoiesis in a mammal include hEPO analog, such as human serum albumin fusion proteins described in PCT Publication WO 99/66054 (Genzyme Transgenics Corp), the tutorial content of which is incorporated herein by reference, and EPO mutants, such as described in PCT Publication WO 99/38890 (Beth Israel Deaconess Medical Center), the tutorial content of which is incorporated herein by reference. In the WO 99/38890 publication, an EPO mutant includes an isolated nucleic acid encoding EPO, where the nucleic acid has one or more mutations in a non-coding region and the EPO has altered biological activity. In one embodiment, the mutation is in the 51 non-coding region.

Other ERAs indicated as capable of stimulating erythropoiesis in a mammal include EPO omega, which may be produced from an Apa I restriction fragment of the hEPO gene described in U.S. Pat. No. 5,688,679 (Powell), the tutorial content of which is incorporated herein by reference, and altered glycosylated hEPO, such as described in PCT Publication WO 99/11781 (Hoechst Marion Roussel Deutschland GMBH), the content of which is incorporated herein by reference. In the WO 99/11781 publication, the altered glycosylated hEPO includes a polypeptide having part or all of the primary structural conformation of EPO that is a product of eukaryotic expression of an exogenous DNA sequence.

Another ERA identified as capable of stimulating erythropoiesis in a mammal includes polyethylene glycol (PEG) conjugated erythropoietin analogs described in, for example, PCT Publications WO 98/05363 (Ortho Pharmaceutical Corporation), the tutorial content of which is incorporated herein by reference, and WO 01/76640 (Amgen, Inc.), the tutorial content of which is incorporated herein by reference, and U.S. Pat. No. 5,643,575 (Martinez et al.), the content of which is incorporated herein by reference.

Other examples include cell lines modified for expression of endogenous human EPO as described in PCT Publication WO 99/05268 (Boehringer Mannheim GMBH), the tutorial content of which is incorporated herein by reference, and WO 94/12650 (Transkaryotic Therapies, Inc.), the tutorial content of which is incorporated herein by reference. Tissue and cyto-protective forms of ERAs are also contemplated.

ERAs according to the invention may also include long-acting forms of EPO. As used herein, a “long-acting EPO” includes sustained release compositions and formulations of EPO with increased circulating half-life, typically achieved through modification, such as reducing immunogenicity and clearance rate, and EPO encapsulated in polymer microspheres.

One example of a long-acting EPO is disclosed in PCT publication WO 02/49673 (F. Hoffman-La Roche AG), the content of which is incorporated herein by reference. The WO 02/49673 publication describes a conjugate comprising an erythropoietin glycoprotein having an N-terminal alpha-amino group, chosen from hEPO or its analogs having sequence of hEPO modified by addition of 1-6 glycosylation sites or a rearrangement of a glycosylation site, where the glycoprotein is covalently linked to a PEG group.

Other examples of long-acting EPO include, but are not limited to, PEG-modified EPO disclosed in PCT publication WO 02/32957 (Chugal Seiyaku Kabushiki Kaisha, Japan), conjugates of glycoproteins having erythropoietic activity and having at least one oxidized carbohydrate moiety covalently linked to a non-antigenic polymer disclosed in PCT publication WO 94/28024 (Enzon, Inc.), and other PEG-EPO prepared using succinimidyl carboxymethylated PEG (SCM-PEG), succinimidyl propionate PEG (SPA-PEG), and SBA-PEG.

A particle formulation according to an embodiment of the invention may include 0.1 to 99.9% by weight in total solid, preferably 1 to 30% by weight in total solid, of an ERA. In one embodiment, the ERA in the particle formulation is stabilized against aggregation with a stabilizer and a buffer. In one embodiment, the stabilizer used in the particle formulation includes sugar. The sugar may be present in the particle formulation in an amount ranging from 0.1 to 99.9% by weight. Examples of sugars that may be included in the particle formulation include, but are not limited to, sucrose, trehalose, glucose, lactose, maltose, and fructose. In one embodiment, the buffer used in the particle formulation is present in an amount ranging from 0.1 to 99.8% by weight. Preferably, the buffer has a pH value between 5.0 and 8.0, more preferably between 5.5 and 7.5. In one embodiment, the buffer concentration is in a range from 5 mM to 50 mM in solution. Examples of buffers include, but are not limited to, citrate, histidine, phosphate, succinate, maleate, tris, acetate, carbonate, and gly-gly. Of these examples, citrate and histidine buffers are most preferred. The ratio of stabilizer to ERA can be variable. With citrate buffer, the ratio of stabilizer to ERA is preferably greater than 2.0.

In other embodiments of the invention, the stabilizer used in the particle formulation may include in addition to sugar one or more components selected from the group consisting of amino acids, polyols, and polymers. The particle formulation may include 0 to 99.9% by weight amino acid, 0 to 99.9% by weight polyol, and 0 to 99.9% by weight polymer. Examples of amino acids that may be incorporated in the particle formulation include, but are not limited to, histidine, glycine, alanine, L-leucine, glutamic acid, isoleucine, methonine, L-threonine, 2-pheylamine, and arginine. Examples of polyols that may be incorporated in the particle formulation include, but are not limited to, sorbital and mannitol. Examples of polymers that may be incorporated in the particle formulation include, but are not limited to, polyvinylpyrrolidone (PVP), dextran, and propylene glycol.

The particle formulation may include other excipients selected from, for example, surfactants, bulking agents, and salts. The particle formulation may include 0 to 10 wt %, preferably 0 to 5 wt %, of a surfactant, 0 to 99.9 wt %, preferably 0 to 70 wt %, of a bulking agent, and 0 to 99.9 wt %, preferably 0 to 70 wt %, of a salt. The surfactant included in the particle formulation may be ionic or nonionic. Examples of surfactants include, but are not limited to, polyoxyethylene (20) sorbitan monolaurate (trade name TWEEN® 20), polyoxyethylene sorbitan monooloeate (trade name TWEEN® 80), polyoxyethylene-polyoxypropylene glycol (trade name PLURONIC F68), and sodium docecyl sulfate (SDS). Examples of bulking agents include, but are not limited to, mannitol and glycine. Examples of salts include, but are not limited to, sodium chloride, calcium chloride, and magnesium chloride.

A pre-formulation study was performed to assess the effects of pH, buffer type (citrate, histidine, tris), salt (NaCl), metal complex (zinc acetate and calcium chloride), amino acid (arginine), and sugar (sucrose) on stability of EPO (epoetin alfa) in solution. The stability of EPO in solutions was evaluated using Size Exclusive Chromatography (SEC). The stability was evaluated in terms of total soluble aggregate, which is the percentage of the EPO-related compounds that are larger than monomer and soluble in water.

The following examples are presented for illustration purposes and are not to be construed as limiting the invention as otherwise described herein.

### EXAMPLE 1

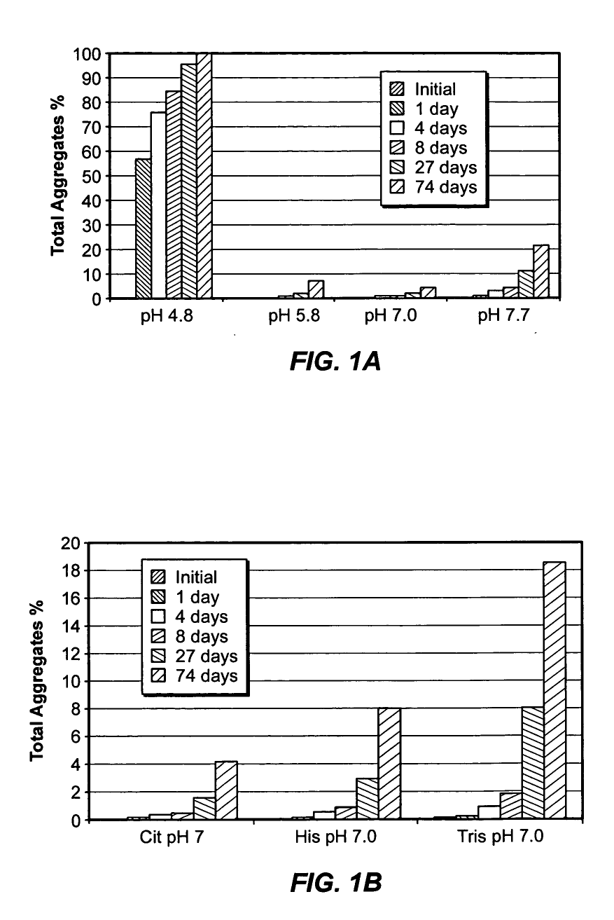

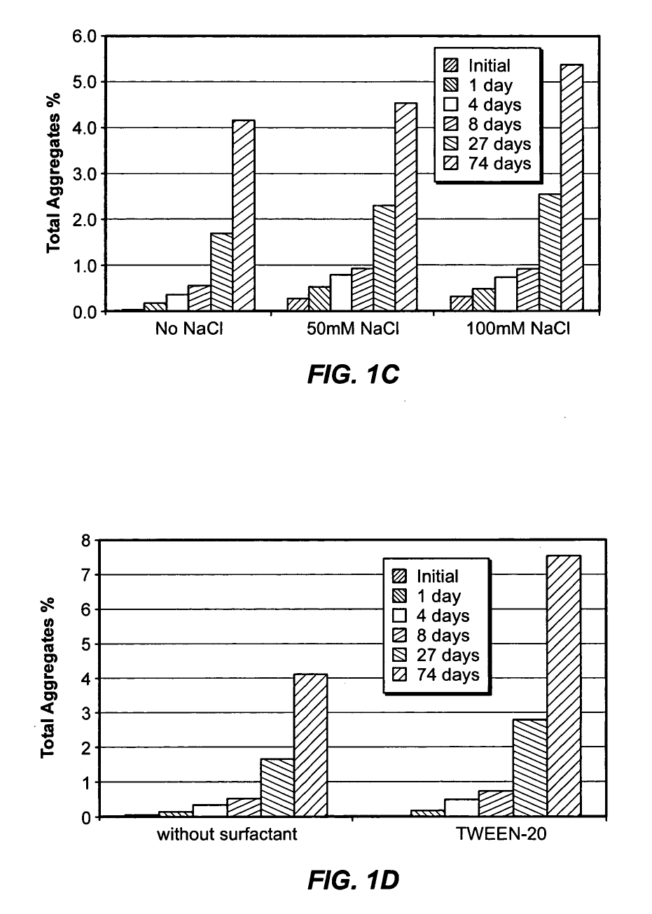

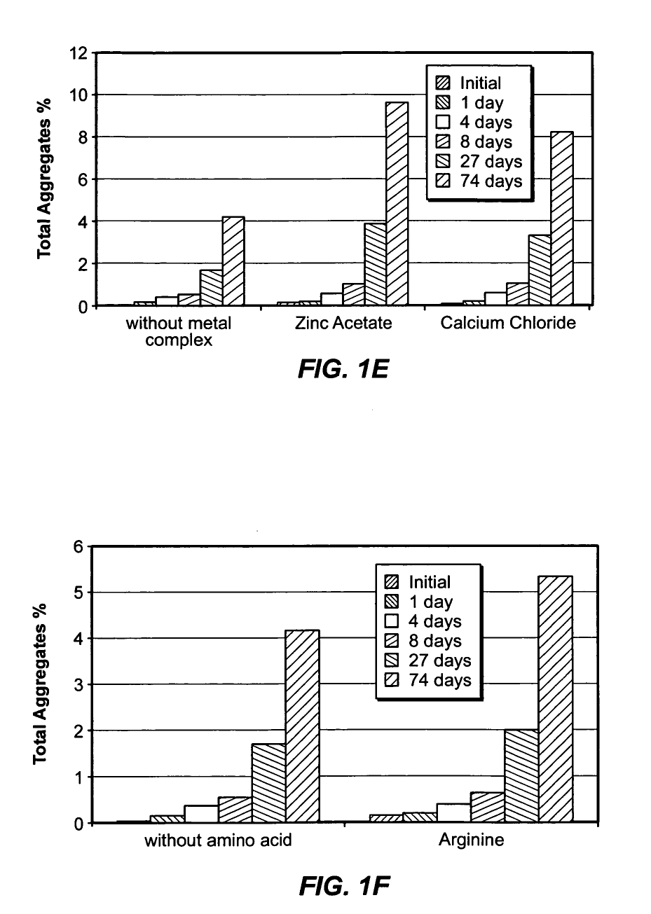

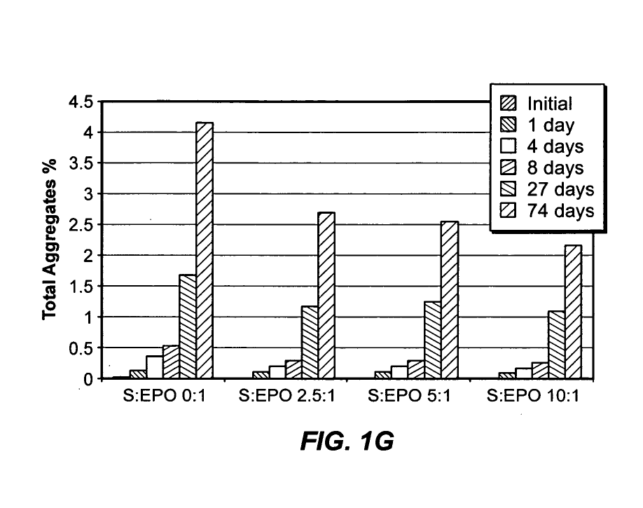

A bulk solution of EPO was obtained as a frozen solution having a concentration of approximately 3.1 mg/ml. Four samples of the EPO solution were dialyzed against buffer solutions to make final solutions having pH of 4.8, 5.8, 7.0, and 7.7, respectively. The stability of EPO in the solutions having pH of 4.8, 5.8, 7.0, and 7.7 was assessed over 74 days at 40° C. The results are shown in FIG. 1A. At 74 days, total soluble aggregate is 100% for the solution having pH of 4.8, less than 10% for the solutions having pH of 5.8 and 7.0, and slightly greater than 20% for the solution having pH of 7.7.

### EXAMPLE 2

A bulk solution of EPO was obtained as a frozen solution having a concentration of approximately 3.1 mg/ml. Three samples of the EPO solution were dialyzed against a citrate buffer, a histidine buffer, and a tris buffer, respectively, each buffer having a pH of 7.0. The stability of EPO in the citrate-, histidine-, and tris-buffered solutions was assessed over 74 days at 40° C. The results are shown in FIG. 1B. At 74 days, total soluble aggregate is slightly greater than 4% with citrate buffer, equal to 8% with histidine buffer, and slightly greater than 18% with tris buffer.

### EXAMPLE 3

A bulk solution of EPO was obtained as a frozen solution having a concentration of approximately 3.1 mg/ml. Three samples of the EPO solution were prepared with NaCl in concentrations of 50 mM and 100 mM added to two of the samples, respectively. The stability of EPO in the solutions was assessed over 74 days at 40° C. The results are shown in FIG. 1C. At 74 days, the total soluble aggregate is slightly greater than 4% without NaCl, about 4.5% with 50 mM NaCl, and about 5.5% with 100 mM NaCl. The results show that total soluble aggregate increases with concentration of NaCl.

### EXAMPLE 4

A bulk solution of EPO was obtained as a frozen solution having a concentration of approximately 3.1 mg/ml. Two samples of the EPO solution were prepared with TWEEN® 20 (surfactant) added to one of the samples in an amount of 0.01 w/v %. The stability of EPO in the solutions was assessed over 74 days at 40° C. The results are shown in FIG. 1D. At 74 days, total soluble aggregate is slightly greater than 4% without surfactant and about 7.5% with surfactant.

### EXAMPLE 5

A bulk solution of EPO was obtained as a frozen solution having a concentration of approximately 3.1 mg/ml. Three samples of the EPO solution were prepared with zinc acetate and calcium chloride (metal complex) added to two of the samples, respectively. The stability of EPO in the solutions was assessed over 74 days at 40° C. The results are shown in FIG. 1E. At 74 days, total soluble aggregate is about 4% without metal complex, about 9.5% with zinc acetate, and about 8% with calcium chloride.

### EXAMPLE 6

A bulk solution of EPO was obtained as a frozen solution having a concentration of approximately 3.1 mg/ml. Two samples of the EPO solution were prepared. One sample contained arginine (amino acid), whereas the other sample did not contain arginine. The stability of EPO in the solutions was assessed over 74 days at 40° C. The results are shown in FIG. 1F. At 74 days, total soluble aggregate is about 4% without arginine and about 5.3% with arginine.

### EXAMPLE 7

A bulk solution of EPO was obtained as a frozen solution having a concentration of approximately 3.1 mg/ml. Four samples of the EPO solution were prepared. Sucrose was added to the samples to make final solutions having sucrose to EPO ratios of 0:1, 2.5:1, 5:1, and 10:1, respectively. The stability of EPO in the sucrose solutions was assessed over 74 days at 40° C. The results are shown in FIG. 1G. At 74 days, total soluble aggregate is about 4.2% with sucrose to EPO ratio of 0:1, about 2.7% with sucrose to erythropoietin ratio of 2.5:1, about 2.6% with sucrose to EPO ratio of 5:1, and about 2.2% with sucrose to EPO ratio of 10:1. The results show that total soluble aggregate decreases as sucrose to EPO ratio increases.

A study was conducted to assess the stability of particle formulations according to embodiments of the invention. The particle formulations were prepared by lyophilization or spray-drying. The stability of the particle formulations was evaluated using SEC. The stability was evaluated in terms of EPO loading and total soluble aggregate. EPO loading is the percent of total soluble EPO in the lyophilized or spray-dried formulation including monomer, dimer, and other higher molecular weight products. EPO loading provides some information about whether or not there are significant amounts of insoluble proteins formed during storage. The total soluble aggregate is the percentage of the EPO-related compounds that are larger than monomer and soluble in water.

For the study, a bulk solution of EPO was obtained as a frozen solution having a concentration of approximately 3.1 mg/ml. Different samples of the EPO solution were dialyzed against a buffer solution. A stabilizer and optionally a surfactant were added to the dialyzed EPO solution to make final erythropoietin to stabilizer to surfactant in a desired ratio. The solution was lyophilized according to the lyophilization cycle shown in Table 1 below.

TABLE 1

| | | Shelf Temperature | | | |
|---|---|---|---|---|---|
| No. | Step | Set Point (° C.) | Rate (° C./min) | Time | |
| 0 | Load Vials | Room temperature | | | |
| 1 | Shelf cool | 4 | 2.5 | ˜7 | min |
| 2 | Hold | 4 | — | 30 | min |
| 3 | Shelf cool | −50 | 2.5 | ˜22 | min |
| 4 | Hold | −50 | — | 3 | hours |

Vacuum Applied

| | | | | | |
|---|---|---|---|---|---|
| 5 | Shelf heat | −20 | 0.14 | ˜3.6 | hours |
| 6 | Hold | −20 | — | 24 | hours |
| 7 | Shelf heat | −15 | 0.14 | ˜36 | min |
| 8 | Hold | −15 | — | 24 | hours |
| 9 | Shelf heat | 0 | 0.14 | ˜107 | min |
| 10 | Hold | 0 | — | 10 | hours |
| 11 | Shelf heat | 20 | 0.14 | ˜2.5 | hours |
| 12 | Hold | 20 | — | 12 | hours |
| 13 | Shelf heat | 30 | 0.14 | ˜72 | min |
| 14 | Hold | 30 | — | 4 | hours |
| 15 | Shelf cool | 4 | 2.5 | 10 | min |

The following examples are presented for illustration purposes and are not to be construed as limiting the invention as otherwise described herein.

### EXAMPLE 8

Ten lyophilized formulations were prepared as described above with citrate as the buffer, sucrose as the stabilizer, and TWEEN® 20 as the surfactant. Table 2 below shows the lyophilized formulations.

TABLE 2

| | | | TWEEN ® | Citrate, mM in |
|---|---|---|---|---|
| | Sucrose:EPO | EPO loading | 20 | solution before |
| Formulation | Ratio | (wt %) | (wt %) | lyophilization |
| A | 2.5 | 17.8 | 1 | 25 |
| B | 2.5 | 18.0 | 0 | 25 |
| C | 13.5 | 6.0 | 1 | 25 |
| D | 13.5 | 6.0 | 0 | 25 |
| E | 2.5 | 25.6 | 0 | 5 |
| F | 13.5 | 6.6 | 1 | 5 |
| G | 8 | 9.7 | 0.5 | 15 |
| H | 13.5 | 6.7 | 0 | 5 |
| I | 2.5 | 24.6 | 1 | 5 |
| J | 4.2 | 16.5 | 0.5 | 10 |

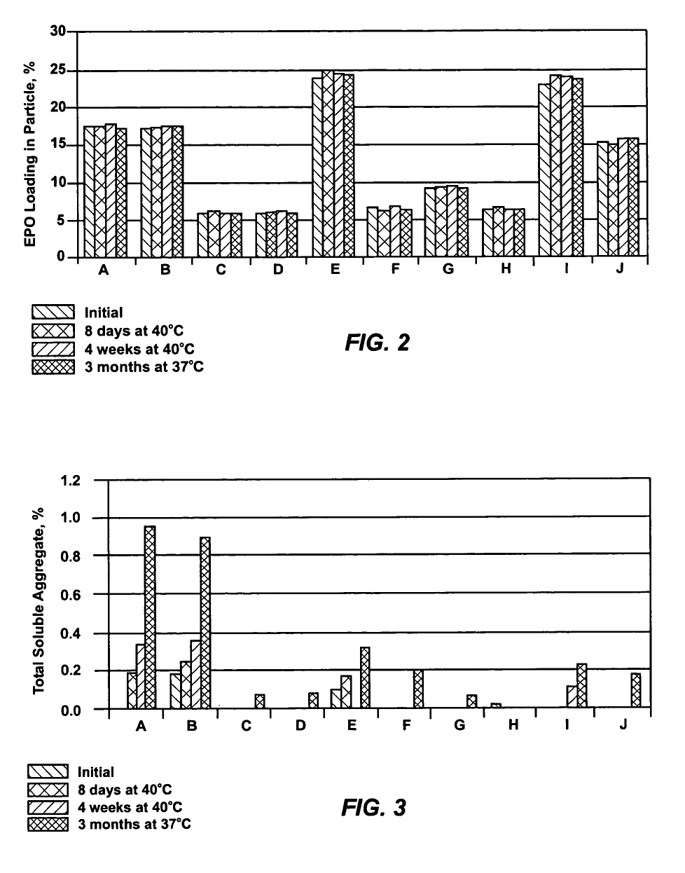

The lyophilized formulations shown in Table 2 were stored at 40° C. for 4 weeks and at 37° C. for three months. Table 3 below shows EPO loading at initial, 8 days and 4 weeks at 40° C., and 3 months at 37° C. The EPO loading at these stability points are also depicted in FIG. 2. The results show that there is no trend of decrease in EPO loading when the formulations are stored at 40° C. for 4 weeks and at 37° C. for three months, indicating there was no formation of significant amount of insoluble proteins.

TABLE 3

| | | | | 3 months at |
|---|---|---|---|---|
| Formulation | Initial | 8 days at 40° C. | 4 weeks at 40° C. | 37° C. |
| A | 17.5 | 17.4 | 17.8 | 17.2 |
| B | 17.3 | 17.5 | 17.6 | 17.5 |
| C | 5.84 | 6.08 | 5.90 | 5.94 |
| D | 5.83 | 6.02 | 6.02 | 5.89 |
| E | 23.8 | 24.9 | 24.8 | 24.5 |
| F | 6.53 | 6.49 | 6.55 | 6.46 |
| G | 9.08 | 9.46 | 9.31 | 9.10 |
| H | 6.42 | 6.56 | 6.49 | 6.37 |
| I | 23.1 | 24.2 | 24.2 | 24.0 |
| J | 15.2 | 15.4 | 15.8 | 15.7 |

Table 4 below shows total soluble aggregate at initial, 8 days and 4 weeks at 40° C., and 3 months at 37° C. The total soluble aggregate at these stability points are also depicted in FIG. 3. Formulations C, D, E, F, G, H and J had 0% total soluble aggregate when stored for 4 weeks at 40° C. Formulations C, D, G, and H had less than 0.1% total soluble aggregate when stored at 37° C. for 3 months.

TABLE 4

| | | 8 days at | 4 weeks at | 3 months at |
|---|---|---|---|---|
| | Initial | 40° C. | 40° C. | 37° C. |
| A | 0 | 0.19 | 0.34 | 0.95 |
| B | 0.19 | 0.25 | 0.36 | 0.90 |
| C | 0 | 0 | 0 | 0.07 |
| D | 0 | 0 | 0 | 0.08 |
| E | 0.10 | 0.17 | 0 | 0.32 |
| F | 0 | 0 | 0 | 0.20 |
| G | 0 | 0 | 0 | 0.08 |
| H | 0.02 | 0 | 0 | 0.0 |
| I | 0 | 0 | 0.11 | 0.24 |
| J | 0 | 0 | 0 | 0.18 |

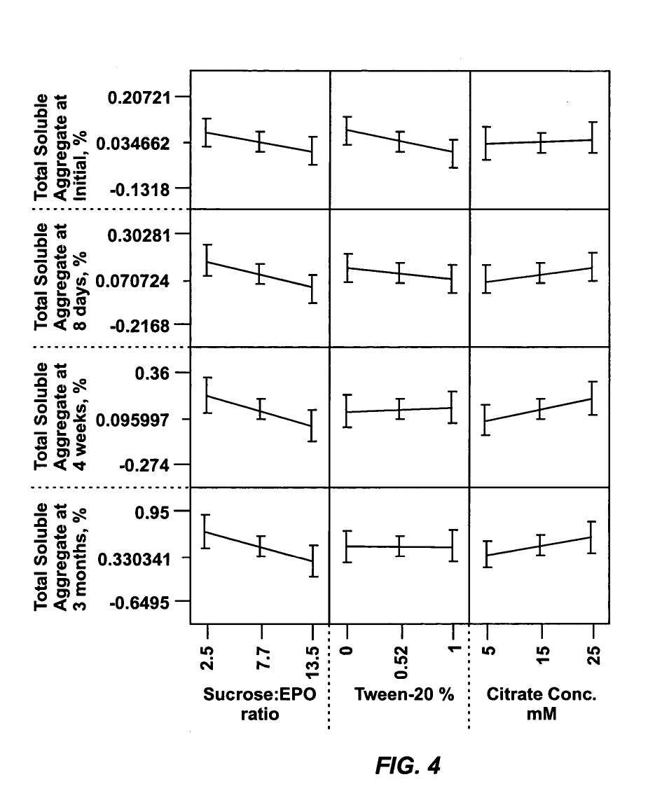

The effects of sucrose to erythropoietin ratio and TWEEN® 20 and citrate concentrations on total soluble aggregate were analyzed using a statistical analysis software. The result of the analysis is shown in FIG. 4. The result shows that higher sucrose to EPO ratio stabilizes EPO formulation during lyophilization and storage at 40° C. Addition of TWEEN® 20 reduces formulation aggregation during lyophilization but does not improve stability during storage at 40° C. Citrate concentration has little effect on EPO stability during lyophilization. However, low citrate concentration shows better stability during storage at 40° C.

### EXAMPLE 9

Six lyophilized formulations were prepared as described above with histidine as the buffer, sucrose as the stabilizer, and TWEEN® 20 as the surfactant. Table 5 below shows the lyophilized formulations.

TABLE 5

| | | EPO | | Histidine, mM |
|---|---|---|---|---|
| | Sucrose:EPO | loading | | solution before |
| Formulation | Ratio | (wt %) | Tween-20 (wt %) | lyophilization |
| K | 13.5 | 6.3 | 1 | 25 |
| L | 13.5 | 6.8 | 0 | 5 |
| M | 2.5 | 26.4 | 1 | 5 |
| N | 2.5 | 21.1 | 0 | 25 |
| O | 8 | 10.2 | 0.5 | 15 |
| P | 13.5 | 17.4 | 0.5 | 10 |

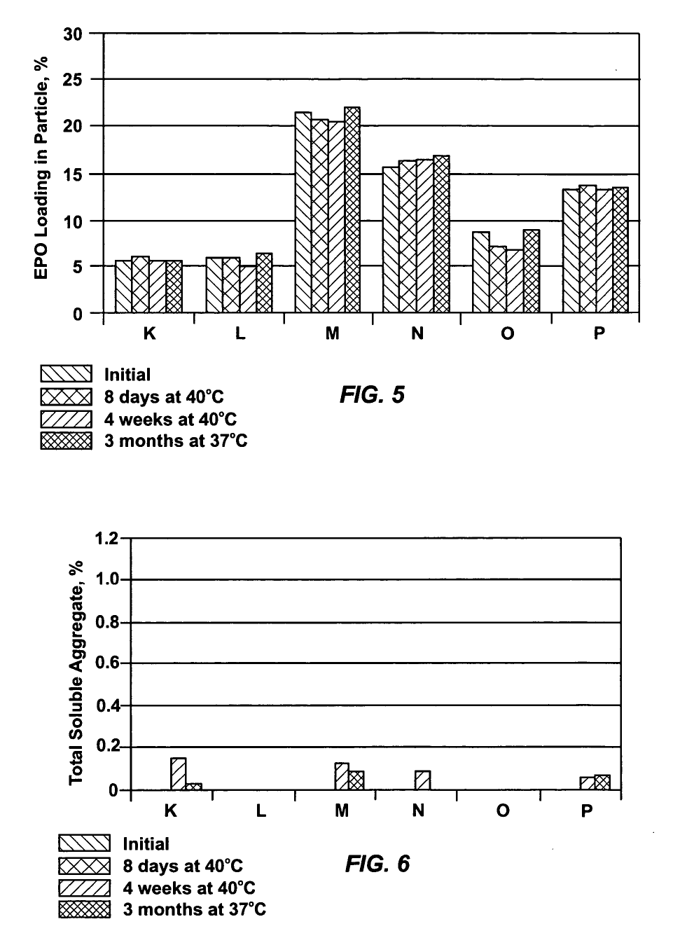

The lyophilized formulations were stored at 40° C. for 4 weeks and at 37° C. for three months. Table 6 below shows EPO loading at initial, 8 days and 4 weeks at 40° C., and 3 months at 37° C. The EPO loading at these stability points are also depicted in FIG. 5. The results show that there is no trend of decrease in EPO loading when the formulations are stored at 40° C. for 4 weeks and at 37° C. for three months.

TABLE 6

| | | | | 3 months at |
|---|---|---|---|---|
| Formulation | Initial | 8 days at 40° C. | 4 weeks at 40° C. | 37° C. |
| K | 5.57 | 5.81 | 5.48 | 5.51 |
| L | 5.85 | 5.82 | 4.92 | 6.17 |
| M | 21.7 | 20.7 | 20.5 | 22.0 |
| N | 15.6 | 16.4 | 16.4 | 16.7 |
| O | 8.57 | 7.18 | 6.66 | 8.87 |
| P | 13.1 | 13.7 | 13.2 | 13.3 |

Table 7 below shows total soluble aggregate at initial, 8 days and 4 weeks at 40° C., and 3 months at 37° C. The total soluble aggregate at these stability points are also depicted in FIG. 6. For all the histidine formulations, total soluble aggregate is less than 0.2% when stored for 4 weeks at 40° C. and for 3 months at 37° C. Formulations L and 0 show 0% total soluble aggregate when stored for 4 weeks at 40° C. and 3 months at 37° C.

TABLE 7

| | | 8 days at | 4 weeks at | 3 months at |
|---|---|---|---|---|
| | Initial | 40° C. | 40° C. | 37° C. |
| K | 0 | 0 | 0.15 | 0.03 |
| L | 0 | 0 | 0 | 0 |
| M | 0 | 0 | 0.13 | 0.09 |
| N | 0 | 0 | 0.09 | 0 |
| O | 0 | 0 | 0 | 0 |
| P | 0 | 0 | 0.06 | 0.07 |

### EXAMPLE 10

A bulk solution of EPO was obtained as a frozen solution having a concentration of approximately 3.1 mg/ml. The EPO solution was dialyzed against 10 mM histidine buffer solution. Sucrose (stabilizer) and TWEEN® 20 (surfactant) were added into the dialyzed EPO solution to make EPO to sucrose to surfactant in a desired ratio. The buffered solution was spray-dried into solid particles having EPO:sucrose:TWEEN® 20:10 mM histidine ratio equal to 1:4.53:0.03:0.50, pH of 6.9, and EPO loading of 16.5%. The spray-dried EPO formulation was stored at 40° C. for 3 months. Three samples were analyzed at initial, 1 month, 2 months, and 3 months using SEC, respectively. At initial time point, the EPO powder had an average particle size of approximately 4.5 μm, a glass transition temperature of 54.9±5.6° C., and a moisture content of 1.16±0.01%. Table 8 below shows the stability results. The results show that the EPO powder is stabilized against aggregation when stored at 40° C. for 3 months.

TABLE 8

| | EPO | | | Total soluble |
|---|---|---|---|---|
| | loading (%) | Monomer (%) | Dimer (%) | aggregate (%) |
| Initial | 15.8 | 100.0 | 0.00 | 0.00 |
| | 16.0 | 100.0 | 0.04 | 0.04 |
| | 16.0 | 100.0 | 0.00 | 0.00 |
| 1 month | 15.6 | 99.9 | 0.091 | 0.09 |
| at 40° C. | | | | |
| | 15.7 | 99.9 | 0.081 | 0.08 |
| | 16.0 | 99.9 | 0.079 | 0.08 |
| 2 months | 15.8 | 99.9 | 0.13 | 0.13 |
| at 40° C. | | | | |
| | 16.0 | 99.9 | 0.13 | 0.13 |
| | 15.9 | 99.9 | 0.13 | 0.13 |
| 3 months | 14.9 | 99.8 | 0.16 | 0.16 |
| at 40° C. | | | | |
| | 15.2 | 99.9 | 0.13 | 0.13 |
| | 15.6 | 99.8 | 0.15 | 0.15 |

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. A particle formulation comprising:
an erythropoietin receptor agonist;
a buffer; and
a sugar;
wherein the buffer and sugar stabilize the erythropoietin receptor agonist against aggregation.

2. The particle formulation of claim 1, which is stable at 40° C. for at least 1 month.

3. The particle formulation of claim 1, which is stable at 37° C. for at least 3 months.

4. The particle formulation of claim 1, which is lyophilized or spray-dried.

5. The particle formulation of claim 1, wherein the erythropoietin receptor agonist is present in an amount ranging from 0.1 to 99.9% by weight.

6. The particle formulation of claim 1, wherein the erythropoietin receptor agonist is present in an amount ranging from 0.1 to 30% by weight.

7. The particle formulation of claim 1, wherein the erythropoietin receptor agonist is erythropoietin.

8. The particle formulation of claim 1, wherein the sugar is sucrose and the buffer is citrate or histidine.

9. The particle formulation of claim 1, wherein the sugar is sucrose, the buffer is citrate, and the weight ratio of sugar to erythropoietin receptor agonist is greater than 2.0.

10. The particle formulation of claim 1, wherein the buffer has a pH value in a range from 5.0 to 8.0.

11. The particle formulation of claim 1, wherein the buffer has a pH value in a range from 5.5 to 7.5.

12. The particle formulation of claim 1, wherein the buffer is present in an amount ranging from 0.1 to 99.8% by weight.

13. The particle formulation of claim 1, further comprising a surfactant in an amount up to 10% by weight.

14. The particle formulation of claim 1, further comprising one or more components selected from the group consisting of surfactants, bulking agents, and salts.

15. The particle formulation of claim 1, further comprising a stabilizing component selected from the group consisting of amino acids, polyols, and polymers.

16. The particle formulation of claim 1, which has a total soluble aggregate less than 3% over 3 months at 37° C.

17. The particle formulation of claim 1, which has dimer less than 3% over 3 months at 40° C.

18. A particle formulation comprising:
an erythropoietin receptor agonist;
a buffer selected from the group consisting of citrate and histidine; and
a sugar;
wherein the particle formulation has a total soluble aggregate less than 3% over 1 month at 40° C.

19. The particle formulation of claim 18, wherein the sugar is sucrose.

20. The particle formulation of claim 18, further comprising a surfactant in an amount up to 10% by weight.
